(19) United States
(12) Patent Application Publication
(10) Pub. No.: US 2019/0209215 A1
(43) Pub. Date: Jul. 11, 2019

(54) Locking cervical plate

(71) Applicant: Atlas Spine, Inc., Jupiter, FL (US)

(72) Inventors: Matthew G. Baynham, Jupiter, FL (US); Brett Zarda, Jupiter, FL (US)

(21) Appl. No.: 16/240,206

(22) Filed: Jan. 4, 2019

Related U.S. Application Data
(60) Provisional application No. 62/613,935, filed on Jan. 5, 2018.

(51) Int. Cl. A61B 17/70; A61B 17/86; A61B 17/80

(52) U.S. Cl. CPC A61B 17/7059; A61F 2/442; A61B 17/8023; A61B 17/8625

(57) ABSTRACT

A triangular shaped locking cervical plate bounded by three side walls terminating in corners having an aperture adapted to receive a bone screw. A recess is formed in an upper surface of the plate which includes a recess with a continuous sidewall interconnecting each aperture. A locking element is rotatably attached to the cervical plate having two tangs extending outwardly a first distance and a third tang extending outwardly a second distance. The locking element including the tangs are constructed and arranged to fit within the recess. Each aperture is open to receive a bone screw when the locking element is rotated into an unlocked position. Each end of the tang overlays a portion of the bone screw placed within the aperture when the locking element is rotated into a locked position.

### PRIORITY CLAIM

In accordance with 37 C.F.R. § 1.76, a claim of priority is included in an Application Data Sheet filed concurrently herewith. Accordingly, the present invention claims priority to U.S. Provisional Patent Application No. 62/613,935, entitled “LOCKING CERVICAL PLATE”, filed Jan. 5, 2018. The contents of which the above referenced application is incorporated herein by reference.

### FIELD OF THE INVENTION

This invention relates to the field of orthopedic surgery and, particularly, to the area of spinal implants for stabilizing the spatial relationship of vertebrae.

### BACKGROUND OF THE INVENTION

The spine consists of vertebrae that are categorized into sections known as the cervical, thoracic and lumbar sections in a flexible arranged column. The vertebrae are separated by small cartilaginous cushions known as intervertebral discs. Intervertebral discs are oblate spherical structures that maintain the space between adjacent vertebrae. Each intervertebral disc consists of an outer annulus fibrosus, which surrounds the inner nucleus pulposus. The annulus fibrosus consists of several layers of strong annular fibrocartilage to contain the nucleus pulposus and distribute pressure evenly across the disc, wherein a mucoprotein gel serves to absorb shocks.

Deterioration of an intervertebral disc results in limited mobility and can cause severe pain. For instance, normal aging causes the nucleus pulposus to lose fluid and contract in volume, resulting in a reduction in the intervertebral space. Any reduction of space between adjacent vertebrae may put pressure on the nerves of the spinal column. Further, a reduction in volume of the nucleus pulposus reduces the disc's ability to absorb shock, which can result in disc herniations.

Spinal plates are well known in the orthopedic art for fixing bones or bone fragments in a pre-selected spatial orientation. The plates are usually attached to the bones or bone fragments by bone screws designed to make a secure and long lasting connection. The coupling of a bone screw to the bone presents problems of stability and long term usage. A particular problem occurs with bone screws that back-out.

Bone screw back-out takes place when the bone screws attaching a plate to a vertebral body loosen over time. Severe back-out results in the bone screw working itself out of the bone and plate, resulting in instability of the bone or joint. This situation results in increasing pain and additional surgery to correct. Unfortunately, bone screw back-out can lead to the destruction of the bone surrounding the screw, causing instability and making bone screw reattachment problematic. There may be several reasons for the back-out, but normal anatomical stresses from body movements contribute to the problem.

Prior art devices address various back-out situations using various techniques. U.S. Pat. Nos. 6,454,771; 6,730,127; and 6,398,783 disclose bone plates for cervical fixation using a locking screw mechanism to overlay the screw heads. The locking element does not simultaneously cover three bone screws set at different distances from the locking element.

U.S. Pat. No. 6,916,320 discloses an anterior cervical plate system having a locking element with a head formed to lock bone screws to the plate.

U.S. Pat. No. 6,436,142 discloses a lag screw which can expand with the insertion of an expansion screw.

U.S. Pat. No. 6,342,055 discloses a bone plate with bone screws having a snap-in retainer securing the heads to the plate.

U.S. Pat. No. 6,306,139 discloses a spinal plate with an anti-extraction device that rotates over the bone screws. Arms from a rotating flap exceed the side walls of the spinal plate to expose bone screw apertures during installation.

U.S. Pat. No. 6,231,610 discloses a bone plate with diverging bone screws and serrations on the plate to increase holding power.

U.S. Pat. No. 6,224,602 discloses a bone plate with multiple bone screw holes which may be covered by a sliding locking plate. The bone plate has an undercut channel to hold the locking plate in contact with the screw heads. The locking plate is held to the plate by a locking screw once it is slid to the desired position.

U.S. Pat. No. 5,603,713 discloses an anterior lumbar plate attached by screws with various angular connections to the spine.

U.S. Pat. Nos. 7,438,715 and 7,662,174 disclose a spinal plate employing wedge shoes to secure bone screws in position.

U.S. Application Publication No. 2004/0102773 uses the ends of the bone plate to cover the heads of the bone screws.

U.S. Pat. No. 6,740,088 uses extra set screws to interfere with the heads of the bone screws.

What would benefit the industry is an improved triangular shaped cervical bone plate providing attachment using three bone screws, wherein the bone screws can be locked in position with the same locking mechanism when the bone screws are placed at different distances from the locking mechanism.

### SUMMARY OF THE PRESENT INVENTION

A locking cervical plate having an upper surface and a bottom surface bounded by three side walls terminating in corners, each corner having an aperture adapted to receive a bone screw. The upper surface includes a recess section formed from a continuous sidewall and lower surface interconnecting each aperture positioned at each corner. A locking element is rotatably attached to the cervical plate, having two tangs extending outwardly a first distance and a third tang extending outwardly a second distance. The locking element, including the tangs, is constructed and arranged to fit within the recess section to avoid any protrusion from extending outside the side walls of the implant during installation. Each aperture is open to receive a bone screw when the locking element is rotated into an unlocked position. Each end of the tang overlays a portion of the bone screw placed within the aperture when the locking element is rotated into a locked position.

It is an objective of this invention to provide a bone plate with an integral internal screw lock.

A further objective of this invention is to provide a cervical plate having a locking mechanism which engages each bone screw to prevent back out migration of the bone screws.

Another objective of this invention is to provide a cervical plate using three bone screws, two of which are placed at different positions than a third bone screw, with the bone screws retained in position by a single rotating locking element.

Still another objective of this invention is to provide a cervical plate having a locking mechanism that is operated to simultaneously retain bone screws placed at different distances from the center of the locking element.

It is an objective of this invention to provide a cervical plate adapted to span the intervertebral space having bone screw countersunk apertures placed in a triangular position for accepting the heads of bone screws.

Still another objective of the invention is to provide a three sided cervical plate wherein two of the three side walls are equal in length.

Another objective of the invention is to provide a locking cervical plate wherein each end of a tang formed on the locking mechanism is spaced apart at different distances.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE DRAWINGS

FIG. 1 is a perspective and exploded view;

FIG. 2 is a perspective view in an unlocked position; and

FIG. 3 is a perspective view in a locked position.

### DETAILED DESCRIPTION OF THE INVENTION

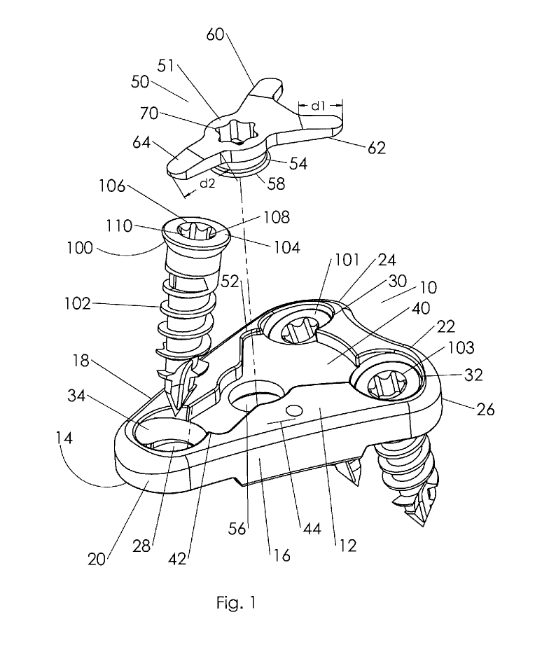

Referring now to FIG. 1, illustrated is the locking cervical plate 10 of the instant invention. The locking cervical plate employs a substantial triangular plate 10 having an upper surface 12 and a bottom surface 14. A first sidewall 16 is bounded to a second sidewall 18 by corner 20. The second sidewall 18 is bound to a third sidewall 22 by corner 24. The third sidewall 22 is bounded to the first sidewall 16 by corner 26. Each of the three side walls terminate in a corner, each corner having an aperture adapted to receive a bone screw; corner 20 having aperture 28, corner 24 having aperture 30, and corner 26 having aperture 32.

Bone screw 100 is depicted having a threaded shank 102 with a head 104. An upper surface 106 of the head 104 is flat with a fastener receptacle 108 for use in rotating the shank 102 into bone purchase. A lower surface 110 of the head 104 is constructed and arranged to interface with countersunk wall 34, depicted with aperture 28. The countersunk wall allows the head 104 of the bone screw 100 to reside flush within the aperture so that the upper surface 106 of the head 104 is flush with the lower surface 40 of the recess section 42 of the cervical plate. For clarity purposes, only a single bone screw 100 is enumerated, and it is to be understood that the other two apertures 30 and 32 include a similar countersunk surface for receipt of a similar bone screw 101 and 103.

The recess section 42 of the cervical plate has a lower surface 40 which is recessed from an upper surface 44. Critical to this invention is the recess section 42 being formed within the three sidewalls 16, 18 and 22. The recess section 42 protects the locking element 50 by containment with the continuous inner sidewall 52 that interconnects the apertures 28, 30 and 32 positioned at each corner 20, 24, and 26.

The locking element 50 is rotatably attached to the plate 10, having a hub 54 that fits through mounting aperture 56. A lower portion 58 of the hub 54 is deformable under pressure, providing a rivet type connection. Once the hub 54 is inserted into the mounting aperture 56, the lower portion 58 is deformed, as illustrated, forming a lip to prevent the hub from removal. From the hub 54, tangs 60 and 62 extend outwardly a first distance d1, and a third tang 64 extends outwardly from the hub 54 a second distance d2. The tangs are constructed and arranged to fit within the recess section 42. The upper surface 51 of the locking element 50 includes a fastener receptacle 70 to allow rotation of the locking element 50. In the preferred embodiment, the fastener receptacle is a torx drive.

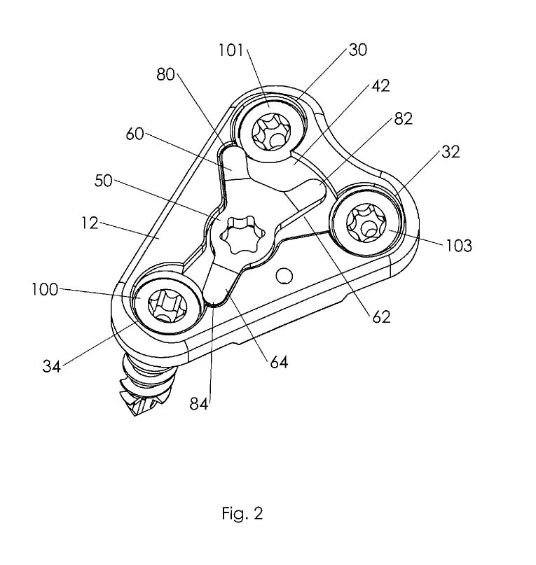

Referring to FIG. 2, illustrated is the plate 12 having the locking element 50 attached within the recess section 42. In this illustration, the end 80 of tang 60 is clear of aperture 30, end 82 of tang 62 is clear of aperture 32, and the end 84 of tang 64 is clear of aperture 34. This allows the bone screws 100, 101 and 103 to be inserted for installation or removed for disassembly. It is noted that the locking element 50 is contained within the recess section 42. In this position, each aperture is open to receive a bone screw when the locking element is rotated into an unlocked position.

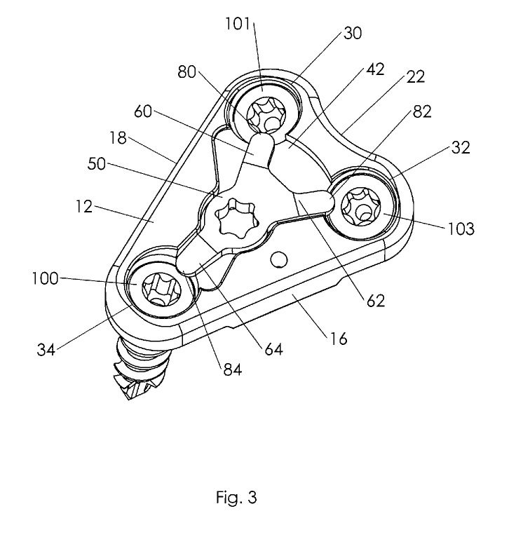

Referring to FIG. 3, illustrated is the plate 12 having the locking element 50 attached within the recess section 42. In this illustration, the end 80 of tang 60 overlaps a portion of aperture 30, end 82 of tang 62 overlaps a portion of aperture 32, and the end 84 of tang 64 overlaps a portion of aperture 34. This allows the bone screws 100, 101 and 103 to be held securely in position with the tangs preventing bone screw back-out. In this position, an end of each said tang overlays a portion of the bone screw placed within the aperture when said locking element is rotated into a locked position.

It is noted that the side walls 16 and 18 are of equal length, and side wall 22 is shorter than side walls 16 or 18. This allows for better purchase in a cervical spine implant, wherein the locking element must be able to overlay the bone screws set at different lengths. Further, the side walls can be scalloped as illustrated by side wall 22.

The plate 12 and bone screws 100, 101, 103 may be coated with a material to promote bone growth, e.g., BMP, bone morphogenic proteins, DBM, demineralized bone matrix, any bone marrow aspirate, platelet rich plasma, composite ceramic hydroxyapatite, tricalcium phosphate, glass resin mixtures, resorbable highly purified polylactides/polylactides-co-glycolides and others.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A cervical plate comprising: a triangular shaped plate having an upper surface and a bottom surface bounded by three side walls terminating in corners, each corner having an aperture adapted to receive a bone screw, a recess section formed in said upper surface forming a lower surface interconnecting each said aperture; a Y-shaped locking element having a hub rotatably attached to said recessed section, said locking element further defined by two tangs extending outwardly from said hub a first distance and a third tang extending outwardly from said hub a second distance which is less than said first distance, said tangs constructed and arranged to rotate from an unlocked position allowing insertion of said bone screws into said apertures to a locked position preventing said bone screws from backing out of said apertures;

2. The locking cervical plate according to claim 1 wherein two of said three side walls of said triangular shaped plate are equal in length.

3. The locking cervical plate according to claim 1 wherein each said two tangs are spaced apart from said hub an equal distance, and said third tang is spaced apart from said hub at a distance less than said two tangs.

4. The locking cervical plate according to claim 1 wherein each said aperture includes a countersunk bore wherein a top of each said bone screw is positioned flush with said lower surface of said recess section.
